(19) United States
(12) Patent Application Publication
(10) Pub. No.: US 2010/0047792 A1
(43) Pub. Date: Feb. 25, 2010

(54) LABEL-FREE OPTICAL DETECTION METHOD

(76) Inventors: István Szendrö, Budapest (HU); Emilia Madarász, Budapest (HU); Katalin Erdélyi, Budapest (HU); Balázs Viktor Varga, Szolnok (HU)

Correspondence Address: Szendro Islvan, Kerekgyarto u. 10, Budapest, H-1147, HU

(21) Appl. No.: 12/450,744

(22) PCT Filed: Apr. 9, 2008
(86) PCT No.: PCT/IB2008/051345

§ 371 Date: Oct. 8, 2009

(30) Foreign Application Priority Data
Apr. 10, 2007 (IB) IB2007000925

(51) Int. Cl. C12Q 1/68

(52) U.S. Cl. 435/6

(57) ABSTRACT

The present provides optical sensor based sensitive, label-free binding assay methods and kits for isothermal real-time detection of the binding of specific analytes (such as nucleic acids, proteins and low molecular weight antigenic or receptor binding ligands) present in low amount in different biological samples. In the binding assays of the invention, the analyte is captured at the specifically pretreated solid surface of optical biosensors in specific recognition reactions (such as hybridization, specific protein-protein interactions and receptor-ligand interactions). The specificity of the methods of the invention is further enhanced by a second specific recognition step using a padlock probe comprising an indicator sequence designed to keep the products of a subsequently performed isothermal nucleic acid amplification method (e.g. rolling circle amplification: RCA) anchored on the sensor surface, enhancing thereby the sensitivity of the detection of the specific binding of the analyte occurred on the sensor surface.

### FIELD OF THE INVENTION

The present invention provides optical sensor based sensitive, label-free binding assay methods and kits for isothermal real-time detection of the binding of specific analytes (such as nucleic acids, proteins and low molecular weight antigenic or receptor binding ligands) present in low amount in different biological samples. In the binding assays of the invention, the analyte is captured at the specifically pretreated solid surface of optical biosensors in specific recognition reactions (such as hybridization, specific protein-protein interactions, receptor-ligand interactions, etc). The specificity of the methods of the invention is further enhanced by a second specific recognition step using a padlock probe comprising an indicator sequence designed to keep the products of a subsequently performed isothermal nucleic acid amplification method (e.g. rolling circle amplification: RCA) anchored on the sensor surface, which amplification is used thereby to enhance the sensitivity of the detection of the specific binding of the analyte occurred on the sensor surface. A real-time optical monitoring of the indicator sequence elongation allows for the determination of the kinetics of amplification which, in turn, makes possible the elaboration of algorithms for quantification of the initial amount of the specifically bound analyte.

### DEFINITIONS

The following expressions will be used in the below defined meanings throughout the whole specification and in the appended claims even if the usual meaning of the said expressions used in the art might be slightly different. Therefore, when interpreting the specific features of the invention and construing the appended claims the below defined meaning of the said expressions should always be used.

“Capture molecules” or “receptors” will always mean specific binding molecules covalently bound to the pretreated optical sensor surfaces of the invention irrespective of their molecular type or size (examples of “receptors” in the herein used sense may be, for example, nucleic acid molecules, proteins and low molecular weight molecules capable of being specifically bound such as antigenic molecules and receptor ligands).

“Ligand” in the presently used sense will mean any molecule that is capable of being specifically bound to the capture molecules or receptors covalently attached to the sensor surface (i.e. nucleic acid molecules, proteins and low molecular weight ligands).

### BACKGROUND OF THE INVENTION

Detection and quantification of analytes in complex biological samples are general tasks in both basic and applied research and also in industrial applications. The quantification of rare biological factors, such as short-lived mRNAs or regulatory proteins (for example: non-house-keeping transcription factors), represents special tasks which include several challenging steps as follows:
selective molecular recognition of the rare analyte concerned;
detection of the selective molecular recognition;
elaboration of time- and cost-efficient detection systems;
amplification of the detected signal if necessary;
elaboration of methods for quantification.

Enzymatic amplification of nucleic acid sequences are widely used for detecting bio-molecules in different biological samples including molecular mixtures of RNAs, cDNAs and proteins, as well as histological sections. Nucleic acid analytes can be recognized and therefore selected by direct hybridization with complementary nucleic acid sequences and amplified thereafter. In the case of proteins or mixed co-polymers, oligonucleotide sequences can be conjugated to a recognizing antibody or specific ligand, and nucleic acid pairing can be used (as above) in a second step.

In the known PCR procedures, which are the most widely used enzymatic nucleic acid amplification methods, the chains to be amplified are recognized and selected by hybridizing with specifically designed primers. In traditional PCR, the non-perfect hybridization of primers to various sequences is partly sorted out by high temperature (stringency) washing. Improved specificity of hybridization can be achieved by mis-match sensitive enzymatic reaction as sequence specific cleavage or ligation (Nilsson et al., 1996. Science 265: 2085-2088). Highly specific analyte selection has been achieved by the application of the so-called padlock-ligation techniques, resulting in single stranded circular DNA only upon binding to the properly matching analyte-sequence, and amplification of these DNA circles exclusively.

Several alternatives of the PCR methodology have also been developed, including both thermal cycling and isothermal approaches. Traditional PCR amplification techniques include high temperature (92° C.-96° C.) reaction steps intercalated in order to separate newly synthesized nucleic acid chains from the template. Therefore, such techniques need thermo cycler instruments, thermo stable enzymes, which can maintain function even at high temperature and temperature-independent detection methods, as well. A well-known example of an alternative thermal cycling method is the ligase chain reaction [“LCR”, Wu, et al., Genomics 4: 560-569 (1990)]. In contrast, during isothermal procedures the nucleic amplification takes place at a constant temperature. These techniques can be based, for example, on the amplification of target molecules using one or more RNA polymerases (WO2006081222), isothermal strand displacement of hybridized nucleic acids (USRE39007E) and on partial destruction of primer molecules which have extended over the template molecule (WO2006087574). The discovery of viral polymerases with high displacement activity opened further ways to elaborate amplification-conditions at standard (isothermal) temperature. The circular rolling amplification or rolling circle amplification (RCA) (U.S. Pat. No. 5,854,033; US Patent App. No.:US2004265897; Lizardi et al., 1998. Nat.Genet. 19: 225-232; Banér et al., 1998. Nucleic Acid Res., 26: 5073-5078) and the ramifying viral amplification allowed to avoid thermal cycles. The above-mentioned rolling circle amplification have been elaborated in many forms. Upon hybridization to the target nucleic acid, a circular probe is used to synthesize several complementary copies that are eventually displaced, thus giving rise to the formation of another product, resulting in the amplification of the target sequence.

The detection of the amplified nucleic acid can be accomplished in several ways. Traditional methods include detection by hybridizing to complementary probes labeled by radioactive, enzymatic or fluorescent means. These methods are time and labor intensive and, therefore, methods were proposed for real time and/or label-free detection of nucleic acids. Elongation of nucleic acid chains can now be followed in realistic time using the well-known real-time PCR assays.

In the wide-spread real-time PCR and gene-array techniques, the amplification is followed by incorporating double stranded chain-specific fluorescence probes or labeled nucleotides into the newly synthesized nucleic acids. Signal detection in these cases demands fluorescent labeling of probes and sensitive fluorescence recording instrumentation. While the necessary temperature changes in traditional PCR methodology hindered the elaboration of non-fluorescent assays, isothermal amplification opened ways to elaborate highly sensitive, non-fluorescent recording methods.

An emerging new field for the label-free detection of analytes relates to the use of different sensors or biosensors, more particularly the use of optical sensors. Several types of optical sensors have been developed and described for the detection of biological analytes, for example the so-called surface plasmon resonance (SPR) and the grating coupler waveguide sensor technology (U.S. Pat. No. 4,815,843; U.S. Pat. No. 5,071,248; Tiefenthaler K, Lukosz W. (1989) J Opt Soc Am B. 6:209; Voros et al., (2002) Biomaterials 23: 3699-3710). The signal detected in these methods are usually the change in size of molecules specifically attached to the inert surface of the sensor. This change in size can be either a decrease or an increase, and the actual setup of the underlying bio/chemical reaction determines the type of signal generated.

There have been several attempts to combine the advantages of the above-mentioned different recognition, amplification and detection methods for elaborating sensitive and convenient binding assay methods. For example, US Patent Appl. No. 2004048287 discloses a method where the recognition of single nucleotide polymorphism takes place on the surface of an SPR sensor to provide label-free specific detection. Target nucleic acids are contacted with the probe immobilized on an inert substrate, recognition takes place, and after a reaction with a cleavage agent, the specific cleavage is detected by the optical sensor by means of detecting the decrease in the size of the complex immobilized to the surface of the sensor. It is also disclosed that RCA as an amplification step could be incorporated into the said method to add extra nucleotides to the recognition structure prior to the specific detection. In this case, RCA is proposed as an optional step to increase the size of the complex to be cleaved prior to the detection of cleavage.

In WO05118871, a method is disclosed for detecting nucleic acids by generating mass changes in a ligand on an SPR surface through target-dependent enzymatic reaction. First, the target nucleic acid of interest is recognized by and bound to a first polynucleotide that binds specifically to the nucleic acid target, wherein the first polynucleotide is designed to permit immobilization to an SPR sensor surface. Next, the complex is contacted with reagents suitable to change the mass of the first polynucleotide via an enzymatic reaction only when the nucleic acid target is present in the complex; and then an SPR signal is detected due to the change generated by a mass change of the first polynucleotide bound to the SPR sensor surface. Real time detection of the target nucleic acid sequence is also proposed. This publication pamphlet also proposes to enhance the created mass signal by using RCA for the elongation of the first polynucleotide. However, neither specific teaching nor experimental evidence is put forward to demonstrate the actual elongation of the first polynucleotide, or for the real time detection of the mass change of the first polynucleotide. Even though RCA is proposed as a means for enhancing the binding specific mass change on the surface of the sensor, there is no hint disclosed on how the continuous attachment of the amplified mass on the sensor surface should be ensured despite the strong strand displacement activity of the polymerase enzymes used in the RCA method.

Despite the tremendous research and development effort recently put into the development of sensitive, user friendly specific binding assays, and the combination of the existing binding assay arrangements with the convenient label free optical sensor technology, no sensitive optical sensor based binding assay have been developed so far which would be useful for label free real-time detection and quantification of low concentration analytes present in biological specimens.

The object of the present inventors was, therefore, to develop optical sensor based sensitive, label-free binding assay methods and kits for the isothermal convenient real-time detection and quantification of the binding of specific analytes (such as nucleic acids, proteins and low molecular weight antigenic or receptor binding ligands) present in low amount in different biological samples.

### DETAILED DESCRIPTION OF THE INVENTION

The present invention, by utilizing already known molecular biological methods, such as the specific nucleic acid recognition techniques by padlock probes (Nilsson et al., 1996. Science 265: 2085-2088), the use of nucleic acid conjugated antibodies and the rolling circle amplification (RCA) procedure (Lizardi et al., 1998. Nat. Genet. 19: 225-232; Banér et al., 1998. Nucleic Acid Res., 26: 5073-5078) provides a novel binding assay arrangement for sensitive real-time detection of the binding of specific analytes to optical biosensor surfaces by assuring an analyte binding specific surface-bound on-site chain elongation. The methods and kits of the invention enable for
i. quantitative measurement of defined rare analytes in complex biological samples;
ii. kinetic analyses of biomolecular chain-elongation;
iii. in real-time and
iv. without additional labeling.

In accordance with the above, in one embodiment of the present invention, highly selective padlock recognition and rolling circle nucleic acid amplification is adopted to a sensitive optical sensor surface, where the amplified chains are bound and produce real-time optical signals proportional with chain-elongation. The sensitive label-free real-time recording allows for the kinetic analysis of the amplification and the quantification of the initial amount of the captured analyte.

The invention comprises the following specific features:
A solid, optical sensor surface, which serves as a detector surface for measuring the amount of depositing material in realistic time (real-time), with high sensitivity and without any need for labeling.
The sensor surface is functionalized with reactive NH—, OH, aldehyde or thiol groups for covalent binding of biomolecules.
Specific capture molecules (“receptors”: e.g., nucleic acid probes, antigens, receptor-specific ligands) are covalently bound to the sensor surface, and potential non-specific binding sites are blocked by group-specific reagents. Functionalization steps can be continuously monitored by optical sensing.
Introduction of the analyte mixture at controlled streaming speed into a heat-controlled measuring cuvette, containing the functionalized sensor surface. Establishment of the optimal reaction conditions (temperature, concentrations, streaming through, sheer force, reaction-time).
The binding of “ligands” molecules to “receptors” can be immediately measured if their concentration in the analyte mixture exceeds the detection threshold.
If the ligand concentration is below the threshold, the above sensor system will not detect the binding reaction, even if a few ligands established specific interactions with the receptors.
In order to detect the specific binding of small amount (even 1 molecule) of the ligand, the method of the invention applies an amplifying method, which produces surface-bound material deposition sufficient for detection and being in proportion to the amount of the initially recognized specifically bound analyte and to the advancement of the reaction-time.
Signal amplification starts with a further step of analyte specific padlock-ligation.
Ligation of the 3′ and 5′ padlock-ends is selective for perfect matching, and the following rolling circle amplification will take place only at specifically sorted (circularized) sites.
The elongating signal-amplifying chains are kept on the sensor surface by specifically designed amplifying probes (see FIG. 1). Besides the matching ligation sequences at the 3′ and 5′ ends, the padlock probe carries a 10-15 nucleotide long sequence identical to a fragment in the indicator sequence. Circular amplification of the padlock-probe results in multiple complementary sequence-copies, which will contain a complementary sequence to the indicator fragment, as well. This 10-15 nucleotide sequence is used to hybridize the elongating chain to the initial analyte-binding site on the receptor surface.

According to an advantageous embodiment of the invention (as shown in FIG. 1):

the initially recognized nucleic acid ligand (i.e. the analyte) is specifically bound to the sensor surface by an immobilized capture probe and then
i. a padlock probe is specifically attached to the surface bound analyte, said probe comprising target specific recognition sequences at the 3′ and 5′ ends, a sequence to bind a complementary primer for rolling circle amplification and an additional 15-20 nucleotides long sequence identical (sense) to a sequence present in the bound nucleic acid analyte, and providing an antisense (complementary) binding site for the nascent chains to the analyte;
ii. the two free ends of the specifically fitting (and only the fitting) padlock chains are bound together with ligase enzyme reaction forming thereby a circular nucleic acid chain which can be a subject of circular amplification exclusively on the specifically recognized ligands;
iii. the circularized (i.e. closed circular) padlock probes are amplified at standard (25-37° C.) temperature by added circular polymerase enzyme, in the presence of a primer designed for the padlock amplification and a mixture of label-free desoxy nucleotide triphosphates; and
iv. the on-going padlock amplification takes place on the sensor surface, starting at the sites of original ligand binding, and locked to the surface by the cyclic production of sequences complementary to the captured analyte nucleic acid. Thereby the reaction provides increasing material deposition at the sites of initial analyte capturing for continuous, real-time optical detection.

According to another advantageous embodiment of the invention (see also FIG. 1):

the initially recognized antigenic substance (i.e. the analyte) is specifically bound to the sensor surface by immobilized capture antibodies and then
i. another analyte specific antibodies carrying a 40-60 nucleotides long nucleic acid chain on their non-binding (C-terminal) region are specifically bound to the captured analyte;
ii. padlock probes containing sequences for specific hybridization ligation on the antibody conjugated oligonucleotide for binding rolling circle amplification primer and a sense sequence for the end of the antibody conjugated oligonucleotide are specifically bound to the surface bound antibodies, while the excess padlock probes and non-bound antibodies will be washed out from the reaction space; and
iii. the bound padlock probes will be ligated, then amplified by circular rolling amplification and the elongation of the chain being bond to the antibody conjugated nucleic acid by the synthesized antisense sequence is measured as described above.

Even more specifically, the invention provides label-free, optical sensor based methods for detecting, in a biological sample, the presence and/or the quantity of analytes capable of being specifically bound by specific capture molecules, said method comprising the following steps:
a) providing analyte specific capture molecules immobilized on the surface of an optical sensor capable of detecting the mass of material being specifically bound on its surface;
b) contacting a biological sample with said optical sensor surface in conditions allowing the specific binding of analytes present in said sample to said capture molecules;
c) contacting said optical sensor surface with a rolling circle amplification (RCA) initiator nucleic acid also comprising a specific binding site for the captured analyte molecules and an anchor sequence ensuring the continuous surface binding of the amplification products in conditions allowing the specific binding of said initiator sequence to said captured analytes;
d) contacting said optical sensor surface, in conditions allowing specific binding, with a padlock-probe comprising 3′ and 5′ sequences perfectly complementary to a continuous sequence region comprised in said initiator sequence in a way that hybridization of said padlock-probe to said initiator sequence results in the circularization of said padlock-probe, wherein said padlock-probe also comprises an anchoring sequence region being complementary to another sequence region of said initiator sequence;
e) contacting said optical sensor surface with reagents providing RCA in conditions enabling RCA, whereby the elongating nucleic acid chain resulting from the ongoing RCA remains continuously anchored to the surface of said optical sensor but only in sites where analyte molecules were specifically captured;
f) detecting the change in the mass of the surface bound material caused by the ongoing analyte specific surface anchored RCA and, optionally, recording said change in time;
g) detecting the presence and/or quantity of the analyte present in said biological sample by comparing said detected mass change data with data obtained previously in similar samples using the same assay arrangement in the presence of known amounts of said analyte.

In advantageous methods of the invention, the analyte to be detected is a nucleic acid molecule, a non-nucleic acid molecule or a supramolecular entity such as complexes of cellular macromolecules or viral particles.
In another advantageous method of the invention the analyte to be detected is a nucleic acid sequence also comprising an RCA initiator sequence region and an anchor sequence region whereby step c) above can be omitted.

In a further advantageous method of the invention, wherein the analyte to be detected is a non-nucleic acid molecule, the analyte specific binding site of the RCA initiator sequence is provided by a specific binding partner of said non-nucleic acid molecule being conjugated to said RCA initiator sequence.

The invention further provides reagent kits for performing the methods of the invention, advantageously comprising: an optical sensor surface conjugated with specific capture molecules, a specific padlock-probe and, optionally, an RCA initiator nucleic acid comprising an anchoring region and a specific binding site for the captured analyte molecules, reagents for performing RCA and instructions for performing one or more of the methods of the invention.

The invention also provides in vitro clinical diagnostic methods based on the detection and/or quantification of an analyte by performing the method according to any of claims 1-10.

### BRIEF DESCRIPTION OF THE DRAWINGS

FIG. 1 is a schematic representation of the label free optical detection method of the invention. The drawing demonstrates that the surface bound analyte is specifically recognized by a specially designed anchoring padlock probe, and the anchored rolling circle amplification on the surface of the optical sensor only proceeds in the presence of the analyte.

FIG. 2 shows the building of a DNA-capturing surface on OWLS (optical waveguide light spectroscope) sensor chips. The amino-functionalized surface was reacted with glutaraldehyde (GA) [2.5% (v/v) in 10 mM Tris buffer containing 20 mM Mg²⁺], then washed with the buffer. The surface was then reacted with PAMAM dendromers (Superfect; Quiagen), then washed again with Tris buffer. E.Coli DNA mix (1 ng/μl; Sigma) was added, then washed with the buffer. Washing steps are indicated by dashed arrows.

FIG. 2b shows the coupling of a “padlock” DNA probe to an “initiator” (biotin-pp90) nucleic acid immobilized on the OWLS sensor surface by avidin-biotin binding. The amino-functionalized surface was reacted with NHS-biotin; the biotinylated surface was reacted with avidin. The 90-nucleotide long DNA sequence with a biotin-conjugated terminal nucleotide (biotin-pp90) was added to the avidin-coated surface, and the binding was followed in real-time with OWLS detection. After removal of excess biotin-pp90 with washing (Tris buffer), the surface was reacted with the 90-base nucleotide (“padlock probe”) designed specifically to recognize by its 3′ and 5′ ends a 10-10 nucleotide long sequence in the “initiator” biotin-pp90. The sequence-specific molecular recognition is confirmed by the measurement data shown in this figure.

FIG. 3 shows the results of a representative OWLS assay on the binding and elution of DNA on a dendromer-coated sensor-surface. Genomic E.Coli DNA (Sigma) was used to monitor the concentration-dependent binding to Superfect coated sensor-surfaces. Different concentrations of DNA were introduced in equal (30 μl) volumes of Tris-Mg buffer solutions, at 25° C., at time points indicated by arrows. Each step of DNA-introduction was followed by 20 minutes washing with Tris-Mg buffer. The binding was broken up only by washing at high (>70° C.) temperature. The onset of the 5 minutes long high-temperature washing steps are indicated by dashed arrows. The steep decreases are due to the temperature-sensitivity of the assay. The base-line (obtained after Superfect binding) is shown by the horizontal dashed line.

FIG. 4 shows the binding of DNA fragments of different size on DNA-capturing optical sensor surfaces.
A: mixtures of DNA fragments in different size-ranges were obtained by sonicating genomial E.Coli DNA (Sigma).
B: binding of DNA fragments of different size to the capturing surface carried out on OWLS sensor chips.

FIG. 5 demonstrates the optical detection of binding of random (A) and complementary (B) cDNAs to the same primer sequence on the surface of OWLS sensor chips. The random cDNA contained the four nucleotides in the same proportion, but in a random order. After initial binding, washing with low ionic strength Tris buffer at 50° C. completely removed the random cDNA, while detectable amount of the complementary cDNA remained on the sensor.

FIG. 6 shows the process of isothermal circular amplification, detected by OWLS. The process starts with washing the “padlock”-carrying sensor-surface with phi29 buffer (a buffer for circular amplification) containing the primer (a 20-nucleotide sequence designed to bind to an amplification starting site on the padlock). Excess primer is removed by 40° C. washing with phi29 buffer. Phi29 enzyme and 0,2 mM mixture of deoxy nucleotide-triphosphates (dNTP) are added in phi29 buffer, at 25° C. After a 40-min incubation, the excess emzyme was removed, fresh dNTP solution was added, and the reaction was left to run at 25° C., overnight. The increase in the absorbed mass, which was not washed off with phi29 buffer, indicated a DNA chain-elongation bound to the sensor surface. While washing at 25° C. did not remove material, washing at 65° C. significantly decreased the mass of material on the sensor surface. The heat-sensitive binding corresponded to the expected DNA-DNA binding mediated by hybridisation between the elongating chain and the “docking” sequence in the “initiator” nucleic acid.

FIG. 7 shows the whole process recorded by OWLS, starting from the binding of the “initiator” nucleic acid to the avidin-coated surface until the heat-removal of amplified DNA from the sensor surface.

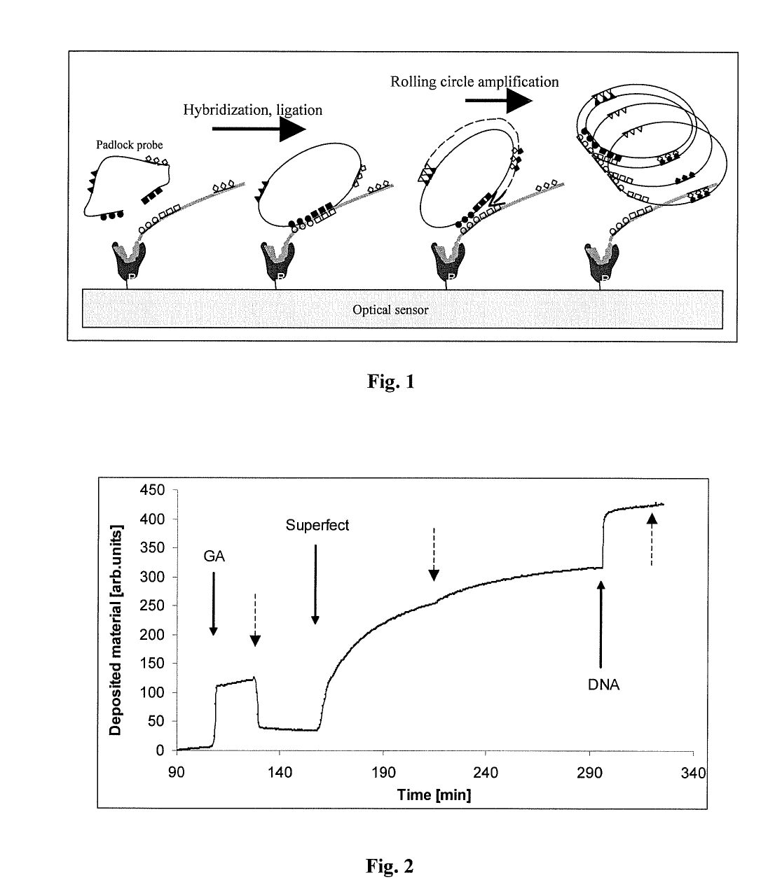

### EXAMPLES

In the following, we will further illustrate the claimed invention by showing experimental examples comprising several advantageous embodiments of the invention. The below examples, however, may not be used anyway for limiting the scope of the invention which, in turn, is defined by the specific features included in the appended claims.

### Example 1

### Functionalization of Optical Sensor Surfaces for DNA Binding

The surface of a sensor-chip of the optical waveguide light spectroscope (OWLS) had been modified by vacuum sylanization or by traditional APTES-reaction in order to produce abundant —NH₂⁺ reactive groups on the sensor-surface, for further chemical reactions as it is explained in detail below.

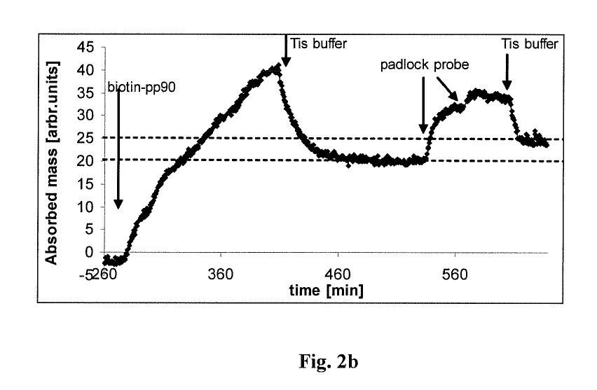

1.1. The amino-moieties were treated with glutaraldehyde, a bifunctional crosslinking molecule. The resulted glutaraldehyde-coated, reactive surface was reacted with PAMAM dendromeres resulting in a sensor-surface carrying a covalently bound lattice of DNA-binding material. (PAMAM dendromeres are materials used for introducing foreign DNA into cells, because of their strong affinity to DNA and three-dimensional particle nature, which triggers cellular endocytosis ( ).) The building of the “capturing” surface had been followed up by continuous monitoring the material deposition by real-time OWLS measurement (FIG. 2)

1.2. The amino-moieties were treated with NHS-biotin to bind biotin molecules covalently to the surface, and the biotinylated surface was reacted with avidin. A 90-nucleotide long DNA sequence with a biotin-conjugated terminal nucleotide (biotin-pp90) was added to the avidin-coated surface and the binding was followed in real-time with OWLS detection. After removal of excess biotin-pp90, the surface was reacted with a 90-base nucleotide (“padlock probe”) designed specifically to contain
i) two sequences at its 3′ and 5′ ends which, together, could complementary cover a confluent 20-base part of biotin-pp90, without any mismatches;
ii) a 20-base long sequence identical to a part of biotin-pp90 designed for docking; and
iii) an amplification starting site to be recognized by a primer designed for initiation of circular amplification.

The binding of the “padlock probe” to the biotin-pp90—functionalized sensor surface was also monitored by OWLS detection (FIG. 2b)

### Example 2

### Binding and Elution of DNA on Functionalized Optical Sensor Surfaces

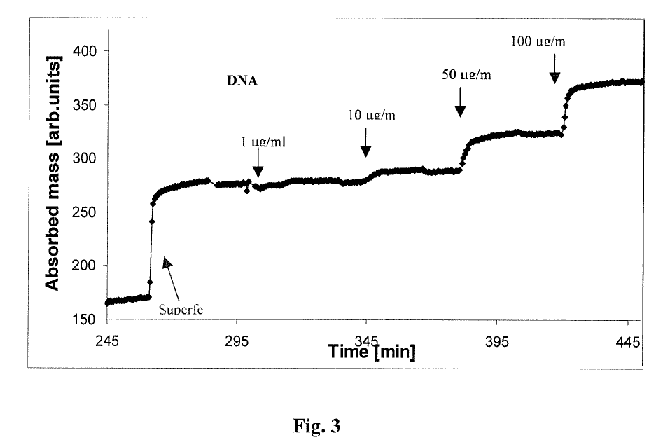

2.1. The dendromer-functionalized sensor surface captured DNA molecules from a mixture of E.Coli-derived DNA (Sigma) (last step on FIG. 2. and FIG. 3) in a concentration-dependent way (FIG. 3). The stability of DNA-binding was probed by washing with a number of different solutions including Tirs+200 mM Mg²⁺, SSC, 1.5 M Sodium-acetate, 0.05 M HCl, 2-10 mM EDTA in Tris buffer (TE). None of the eluting solutions could remove the captured DNA.

The binding could be broken up by washing with low ionic strength solutions at high (>70° C.) temperature (FIG. 3). The conditions for disruption of dendromer-DNA interaction suggest that the binding forces, which stabilize the DNA—dendromer complexes are similar to those coupling DNA double strands together.

High temperature washing, on the other hand, did not remove the capturing Superfect layer (FIG. 3), thus the functionalized sensors could be used repeatedly.

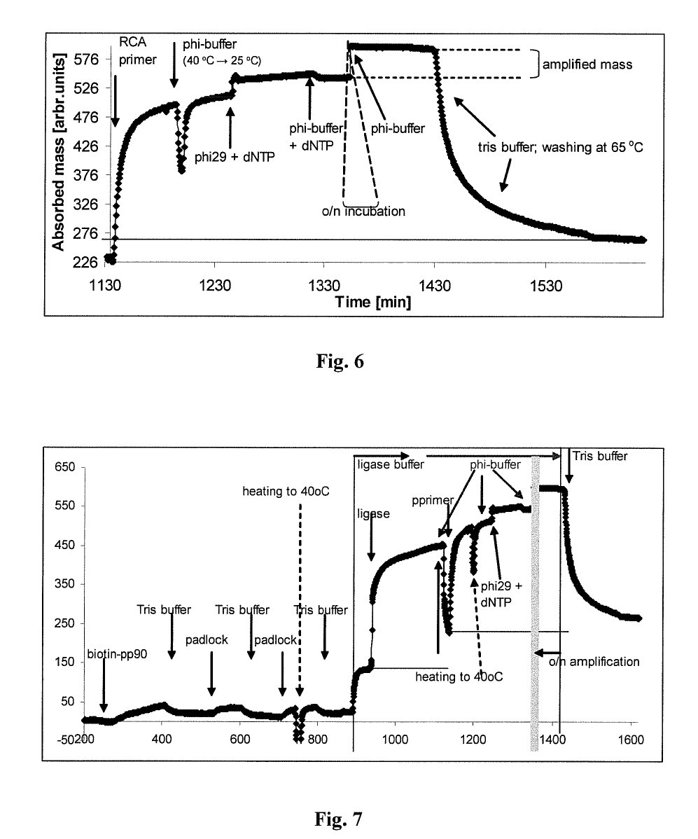

2.2 Functionalization of the sensor surface with biotin provided the advantage that the biotin-avidin coupling was not disrupted by high temperature washing. It allowed using relatively moderate temperature washing steps to remove DNA chains binding with low-stringency. As another advantage, spontaneously forming double-strands of DNA could be melted by heating at variouststeps of the process (see FIG. 7). The temperature was changed by increasing the interior temperature of the OWLS cuvette

### Example 3

### Size-Dependent DNA Binding on Sensor Surfaces

The DNA binding was further analyzed by probing the dendromer-coated sensors with DNA fragments of different size. DNA fractions containing fragments with various lengths were prepared by sonication of the genomic E.Coli DNA (FIG. 4 A).

50 μg/ml solutions of each fraction were loaded into the OWLS cuvette and the binding was determined under identical conditions. The analyses demonstrated that relatively small DNA fragments used for specific hybridization and molecular recognition can be stably bound to the sensor surface (FIG. 4 B).

The experimental data demonstrated that relatively small fragments of DNA, those used for specific biomolecular recognition (as in situ hybridization or PCR amplification) can be stably bound to the sensor surface, and the binding can be followed up by real-time optical detection.

### Example 4

### Sensor-Anchored DNA Fragments Recognize Complementary Sequences

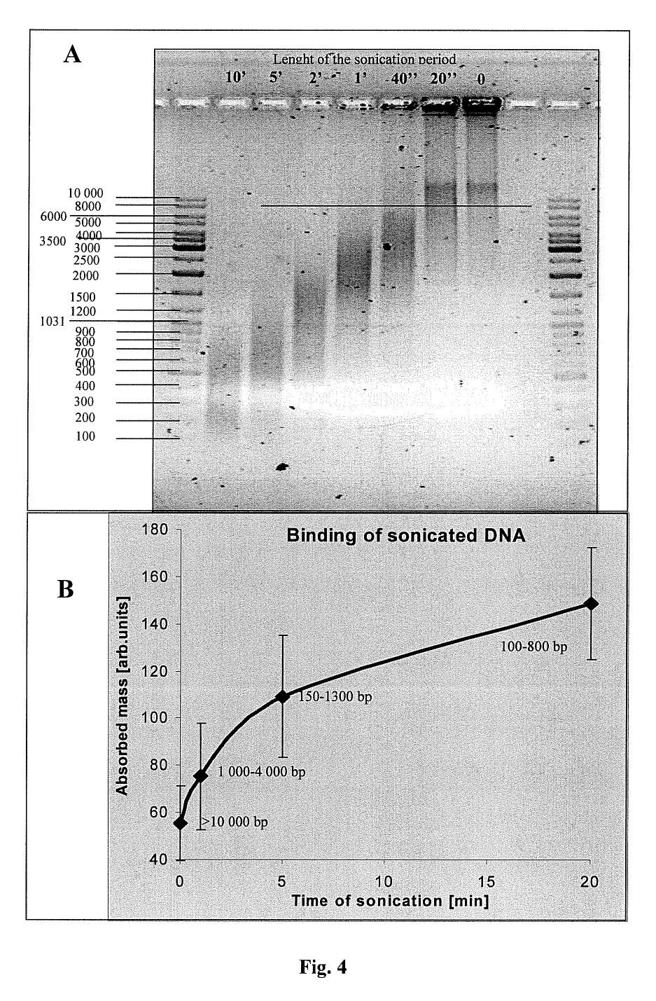

4.1. DNA fragments bound to the dendromer-functionalized sensor surface maintained a structure recognizable by complementary nucleic acid sequences (FIG. 5). To demonstrate the maintenance of bio*recognizable structure, a specific primer for amplification of an Emx2 ( ) gene sequence was bound to the surface and probed either with a cDNA containing the nucleotides in the same proportion but in random order (random cDNA) or with a complementary cDNA of equal length.

4.2. The sequence-specific recognition and binding on biotin-anchored DNA chain (biotin-pp90) was clearly demonstrated by the active ligation of the two ends of the “padlock probe” by T4 ligase. The enzyme (under the applied conditions) can couple chain-ends together only if the ends are hybridized side-to side on a template, without mismatches, According to the resulted efficient amplification in the next steps of the process, the 3′ and 5′ ends of the “padlock probe” could meet on the biotin-pp90 template.

### Example 5

### Isothermal Circular DNA Amplification takes place on the Functionalized Sensor Surface and can be Followed up by real-time OWLS Monitoring

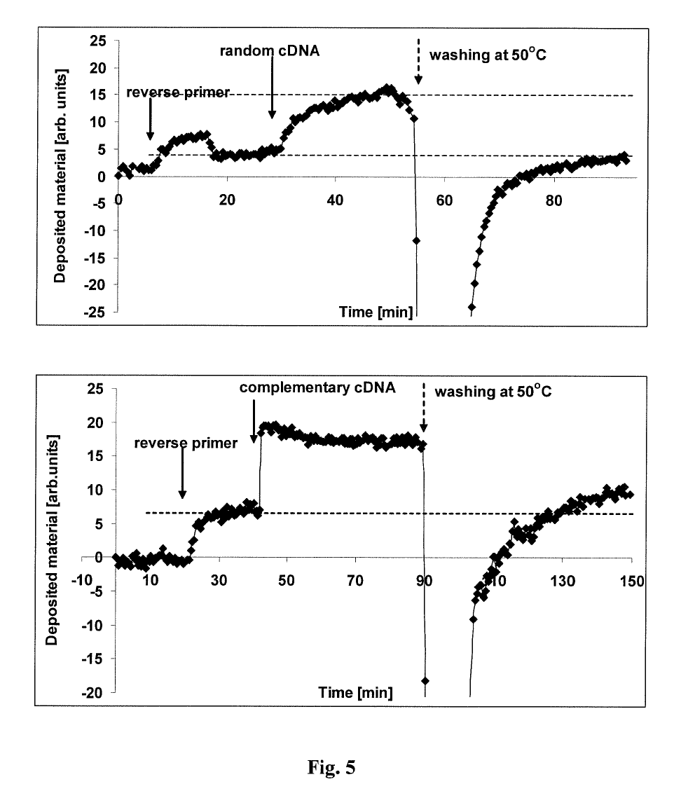

After changing the ligase buffer to a buffer supporting circular amplification by phi29 enzyme, a primer designed for a 20-base sequence of the “padlock probe” was added to the sensor surface carrying the already circular (ligated) padlock-sequence (FIG. 6). After heating to promote specific hybridization of single-stranded form of the primer, the enzyme (phi29), together with a mix of deoxynucleotide-triphosphates (dNTP) was added. The enzyme rapidly bound to the surface, and the reaction seemed to start immediately—as it was indicated by the increase of the “absorbed mass” on the OWLS records. If the reaction was let to run overnight (o/n), a significant increase of the surface-bound material was observed. As it was expected if the amplified chains were coupled to the surface through the hybridization between the “docking sequence” in biotin-pp90 and the complementary sequence amplified from the “padlock probe”, the bound mass was removed by washing at 65° C.

The above description and experimental examples clearly demonstrate the feasibility of the present invention in developing novel user friendly optical sensor based label-free specific binding assays and reagent kits for the detection of different analytes present in low amounts in biological specimens.

What is claimed is:

1. A label-free, optical sensor-based method for detecting, in a biological sample, the presence and/or quantity of analytes capable of being specifically bound by specific capture molecules comprising the following steps:
a) providing analyte specific capture molecules immobilized on the surface of an optical sensor capable of detecting the mass of material being specifically bound on its surface;
b) contacting said optical sensor surface with a biological sample in conditions allowing the specific binding of analytes present in said biological sample to said immobilized capture molecules;
c) providing a nucleic acid comprising a region with a first sequence, to be called “initiator sequence”, complementary to sequences at the 3′ and the 5′ ends of a padlock probe to be applied in a further step, and a region with a second sequence, to be called “anchor sequence”, identical to another sequence of the padlock probe, said nucleic acid
being part of an analyte bound in step b) or
being part of an analyte-specific antibody bound to an analyte in an additional step or
being part of an initiator nucleic acid, for initiation of rolling circle amplification, called “RCA initiator nucleic acid”, comprising a binding site for and being bound to bound analyte molecules or being conjugated to a specific binding partner for an analyte molecule.
d) contacting said optical sensor surface with a padlock probe comprising:
sequences at the 3′ and 5′ ends perfectly complementary to said initiator sequence,
a sequence identical to the “anchor sequence”, and
a sequence to bind a complementary primer for rolling circle amplification under conditions allowing hybridization with that padlock probe;
e) contacting said optical sensor surface with ligase enzyme under conditions that the specifically hybridized 3′ and 5′ ends are bound together, thus forming a circular nucleic acid chain;
f) contacting said optical sensor surface with reagents providing rolling circle amplifications and under conditions enabling rolling circle amplification, whereby the elongating chain resulting from the rolling circle amplification remains continuously anchored to the surface of said optical sensor at produced sequences complementary to said anchor sequence, thus performing surface-anchored isothermal, rolling circle amplification;
g) detecting a change in mass of the surface-bound material caused by the ongoing analyte-specific, surface anchored rolling circle amplification, and recording said change as a function of time; and
h) detecting the presence and/or quantity of the analyte present in said biological sample by comparing said detected change in mass with library data obtained with using the same arrangement with “standard samples” containing known amounts of the pure analyte, e.g. by comparison with a calibration curve for that analyte.

2. The method according to claim 1, wherein the analyte to be detected is a nucleic acid molecule.

3. The method according to claim 1 or claim 2, wherein the analyte to be detected is a nucleic acid sequence also comprising an RCA initiator sequence region and an anchor sequence region whereby step c) is omitted.

4. The method according to claim 1, wherein the analyte to be detected is non-nucleic acid molecule and the analyte specific binding site of the RCA initiator sequence is provided by a specific binding partner of said non-nucleic acid molecule being conjugated to said RCA initiator sequence.

5. The method according to any of claims 1-4, wherein the analyte to be detected is a supramolecular entity such as complexes of cellular macromolecules or viral particles.

6. A label-free, optical sensor based method for detecting, in a biological sample, the presence and/or the quantity of analytes capable of being specifically bound by specific capture molecules comprising the following steps:
a) providing analyte specific capture molecules immobilized on the surface of an optical sensor capable of detecting the mass of material being specifically bound on its surface;
b) contacting a biological sample with said optical sensor surface in conditions allowing the specific binding of analytes present in said sample to said capture molecules;
c) contacting said optical sensor surface with a rolling circle amplification (RCA) initiator nucleic acid also comprising a specific binding site for the captured analyte molecules and an anchor sequence ensuring the continuous surface binding of the amplification products in conditions allowing the specific binding of said initiator sequence to said captured analytes;
d) contacting said optical sensor surface, in conditions allowing specific binding, with a padlock-probe comprising 3′ and 5′ sequences perfectly complementary to a continuous sequence region comprised in said initiator sequence in a way that hybridization of said padlock-probe to said initiator sequence results in the circularization of said padlock-probe, wherein said padlock-probe also comprises an anchoring sequence region being complementary to another sequence region of said initiator sequence;
e) contacting said optical sensor surface with reagents providing RCA in conditions enabling RCA, whereby the elongating nucleic acid chain resulting from the ongoing RCA remains continuously anchored to the surface of said optical sensor but only in sites where analyte molecules were specifically captured;
f) detecting the change in the mass of the surface bound material caused by the ongoing analyte specific surface anchored RCA and, optionally, recording said change in time;
g) detecting the presence and/or quantity of the analyte present in said biological sample by comparing said detected mass change data with data obtained previously in similar samples using the same assay arrangement in the presence of known amounts of said analyte.

7. The method according to claim 6 wherein the analyte to be detected is a nucleic acid molecule.

8. The method according to claim 6 or claim 7, wherein the analyte to be detected is a nucleic acid sequence also comprising an RCA initiator sequence region and an anchor sequence region whereby step c) is omitted.

9. The method according to claim 6, wherein the analyte to be detected is non-nucleic acid molecule and the analyte specific binding site of the RCA initiator sequence is provided by a specific binding partner of said non-nucleic acid molecule being conjugated to said RCA initiator sequence.

10. The method according to claim 6, wherein the analyte to be detected is a supramolecular entity such as complexes of cellular macromolecules or viral particles.

11. A reagent kit for performing the method according to any of claims 1-10 comprising:
an optical sensor surface conjugated with specific capture molecules, a specific padlock-probe and, optionally,
an RCA initiator nucleic acid comprising an anchoring region and a specific binding site for the captured analyte molecules, reagents for performing RCA and instructions for performing the method according to any of claims 1-10.

12. An in vitro clinical diagnostic method based on the detection and/or quantification of an analyte by performing the method according to any of claims 1-10.
